(19) United States
(12) Patent Application Publication
(10) Pub. No.: US 2025/0034079 A1
(43) Pub. Date: Jan. 30, 2025

(54) PROCESS FOR RECOVERING AMINOBENZOIC ACID FROM AN AQUEOUS MOTHER LIQUOR

(71) Applicant: Covestro Deutschland AG, Leverkusen (DE)

(72) Inventors: Jonas Krause, Langenfeld (DE); Maria Francisco Casal, Leverkusen (DE); Wolf Kloeckner, Köln (DE); Andreas Bednarz, Zülpich (DE)

(21) Appl. No.: 18/716,262

(22) PCT Filed: Dec. 16, 2022
(86) PCT No.: PCT/EP2022/086346

§ 371 Date: Jun. 4, 2024

(30) Foreign Application Priority Data
Dec. 20, 2021 (EP) 21215923.0

(51) Int. Cl. C07C 227/42; C07C 227/16

(52) U.S. Cl. CPC C07C 227/42; C07C 227/16

(57) ABSTRACT

The present invention relates to a process for recovering aminobenzoic acid from an aqueous mother liquor which is produced on crystallization of aminobenzoic acid. The process comprises a step (A) of fermenting a suitable fermentable raw material in the presence of microorganisms to form aminobenzoate anions and/or aminobenzoic acid; a step (B) of crystallising aminobenzoic acid at a pH of 3.0 to 4.7, wherein this crystallisation can be carried out during and/or after the fermentation; a step (C) of extracting the aqueous mother liquor of the crystallisation using an alkanol with 8 to 12 carbon atoms to obtain a first alcoholic phase containing aminobenzoic acid and a first aqueous phase; a step (D) of back-extracting aminobenzoic acid from the first alcoholic phase using an aqueous base solution or acid solution to obtain a second aqueous phase containing anions and cations of the aminobenzoic acid and a second alcoholic phase; and a step (E) of crystallising aminobenzoic acid from the second aqueous phase by feeding the latter back into the crystallisation of step (B) or in a separate crystallisation.

The studies that led to the present invention were financially supported by the German Federal Ministry of Food and Agriculture under funding code 22019918.

The present invention relates to a process for recovering aminobenzoic acid from an aqueous mother liquor obtained in a crystallization of aminobenzoic acid. The process comprises a step (A) of fermenting a suitable fermentable raw material in the presence of microorganisms to form aminobenzoate anions (H₂NC₆H₄COO) and/or aminobenzoic acid (H₂NC₆H₄COOH or H₃N⁺C₆H₄COO⁻), a step (B) of crystallizing aminobenzoic acid at a pH of 3.0 to 4.7, where said crystallization can be conducted during or after the fermentation, a step (C) of extracting the aqueous mother liquor from the crystallization with an alkanol having 8 to 12 carbon atoms to obtain a first alcoholic phase containing aminobenzoic acid and a first aqueous phase, a step (D) of re-extracting aminobenzoic acid from the first alcoholic phase with an aqueous base or acid solution to obtain a second aqueous phase containing anions (H₂NC₆H₄COO⁻) or cations (H₃N⁺C₆H₄COOH) of aminobenzoic acid and a second alcoholic phase, and a step (E) of crystallizing aminobenzoic acid out of the second aqueous phase by recycling it into the crystallization from step (B) or in a separate crystallization.

The production of organic acids by fermentation processes has gained particular attention in the recent past. Among the organic acids obtainable by fermentation, aminobenzoic acid should also be emphasized as an economically important product. Aminobenzoic acid finds use, for example, in the production of dyes, odorants or pharmaceuticals. A further example of the use of aminobenzoic acid is the use thereof in the production of aniline by decarboxylation. Aniline in turn is of particular significance as an intermediate in the production of isocyanates.

Fermentative preparation of aminobenzoic acid is described in the literature. Reference is made by way of example to Balderas-Hemandez, V. E. et al., “Metabolic engineering for improving anthranilate synthesis from glucose in Escherichia coli”, Microb. Cell. Fact. 2009, 8, 19 (doi:10.118611475-2859-8-19). The patent literature also includes publications on this topic; see, for example, international patent application WO 2015/124687 A1, which describes the two-stage production of aniline via ortho-aminobenzoic acid as intermediate, and the literature cited therein. Fermentation processes proceed in an aqueous medium and, in the case of preparation of aminobenzoic acid, generally afford aqueous product mixtures (fermentation broths) with a content by mass of aminobenzoic acid in the range from 10.0 g/L to 100 g/L.

Of particular significance is the ortho isomer of aminobenzoic acid, anthranilic acid. In the metabolism of bacteria and yeasts, anthranilic acid is formed in the shikimic acid pathway as a natural intermediate in the synthesis of tryptophan. In the biotechnological production of anthranilic acid, the conversion thereof in the metabolic pathway is reduced or suppressed in order to achieve accumulation in the fermentation medium. Such a concept for biological production of anthranilic acid and the subsequent catalytic conversion thereof to aniline is described in the international patent applications WO 2015/124686 A1 and WO 2015/124687 A1 that have already been mentioned. A possible recombinant microorganism described is the use of bacteria from the families of the corynebacteria or pseudomonads. A more recent application (WO 2017/102853 A1) describes the use of yeasts.

But para-aminobenzoic acid is also of interest. para-Aminobenzoic acid can be synthesized in the metabolism of bacteria and yeasts via the intermediate chorismate, which forms as an intermediate in the shikimic acid pathway. Chorismate is first converted enzymatically to 4-amino-4-deoxychorismate and then by a second enzyme reaction to para-aminobenzoic acid. A concept for biotechnological production of aniline via the intermediate para-aminobenzoic acid is described in international application WO2014171205. One possible recombinant microorganism described here too is the use of bacteria from the family of the corynebacteria.

The fermentative production of aminobenzoic acid affords a highly dilute aqueous product stream. The aminobenzoic acid product of value is predominantly in charged form as the aminobenzoic acid anion in the case of a fermentation in the range from pH 6 to pH 9 (customary pH range in the use of bacteria as microorganisms). After the performance of workup steps that are customary for fermentations, by adjusting the pH to a value close to or at the isoelectric point, the aminobenzoic acid product of value can be precipitated in solid, electrically neutral form. The aminobenzoic acid can then be separated off by filtration for example. The product that has been filtered off is at first obtained in a state with a high water content (“slurry”). This product is then, for example, washed and if required dried (depending on the planned use), or else taken up in a solvent such as aniline or 1-dodecanol (see WO 2015/124687 A1).

The filtration leaves a mother liquor that still contains a significant residual concentration (according to the solubility of aminobenzoic acid under the respective conditions) of aminobenzoic acid and should therefore be freed of this dissolved aminobenzoic acid as quantitatively as possible prior to disposal thereof as wastewater.

International application WO 2015/124687 A1, which has already been mentioned several times, discloses a preferred embodiment in this regard, in which the mother liquor obtained in the crystallization is worked up in order to obtain further aminobenzoic acid (anthranilic acid here). This is accomplished by a sequence composed of an adsorption and a desorption step. The aminobenzoic acid-enriched desorbate obtained is recycled into the crystallization procedure. Yield losses are thus reduced. The adsorption is done on zeolites or activated carbon, and the desorption is done with water of a pH in the range from 5 to 10 or, alternatively, with organic solvents, especially 1-dodecanol. A variant of such a workup by a sequence of an adsorption step and desorption step is described in international application WO 2018/114841 A1. The process disclosed therein is more particularly characterized in that the desorption is conducted in an acidic environment (pH −0.8 to 3.0). Activated carbon is disclosed as a suitable adsorbent. As well as activated carbon, further adsorbents are of course also suitable for the process described in WO 2018/114841 A1. Particular mention should be made of the polymeric adsorbents that are familiar in the specialist field, preferably those based on polystyrene and/or polydivinylbenzene. Typical pore sizes are in the range from 1.5 to 65 nm, for example 4.5 to 10 nm. There are also adsorber types having micro- and macropores. Commercially available products can be found, for example, under the following brand names: Lewatit (e.g. OC 1064 MD PH or AF 5), Macronet (e.g. MN 270, MN 202, MN 100 or MN 102), PuroSorb (e.g. PAD600), AmberSorb (e.g. L493 or 560) and Amberlite (e.g. XAD4). The use of these absorbents (and others, for example activated carbon) is of course not limited to the acidic desorption described in WO 2018/114841 A1; in fact, these are also suitable for basic desorption. If the desorption is effected under acidic conditions, it is advisable to conduct an additional regeneration of the adsorbent with base from time to time (for example after conducting five acidic desorptions). In this way, the adsorber bed is freed of organic impurities.

Processes using a sequence of adsorption and desorption steps have the general disadvantage that very large volumes of adsorber beds are required in the case of production on an industrial scale. Moreover, the materials used for adsorption do not have unlimited regeneratability, and so have to be regularly replaced at least in portions, which incurs costs and solid waste. Neither of the two applications cited discloses a process comprising the extraction of the mother liquor for recycling of aminobenzoic acid dissolved therein.

International patent application WO 2007/088346 A1 describes a process for recovering anthranilic acid from the mother liquor from a crystallization for isolation of anthranilic acid that has been obtained by a Hofmann rearrangement. The sodium carbonate-containing product of the Hofmann rearrangement is adjusted to a pH of 4.2 with sulfuric acid (a), and precipitated anthranilic acid is filtered off (b). The remaining mother liquor is extracted with an organic solvent at a pH of 4.2; suitable organic solvents disclosed are acetic esters (especially ethyl or butyl acetate), ketones (especially 2-butanone) and aromatic hydrocarbons (especially toluene). The organic extract obtained after phase separation (c) is subsequently re-extracted with sodium hydroxide solution (d), with transfer of anthranilic acid as anthranilate anion into the aqueous phase obtained after phase separation (e). This aqueous, anthranilic acid-enriched phase from the re-extraction is combined with the product of the Hofmann rearrangement and sent to the crystallization together therewith. The aqueous, anthranilic acid-depleted phase obtained in the extraction is adjusted to pH 1.5 with sulfuric acid, an organic solvent is added (f), and the mixture is separated into an aqueous phase and an organic phase (g). In this way, organic impurities are transferred to the organic phase. This organic phase is combined with the organic phase from the re-extraction and distilled. The distillate can be recycled into the process as solvent. The distillation residue is incinerated. The aqueous phase from (g) is sent to the wastewater after purification by a Fenton reaction. A particular disadvantage of this process is the relatively high water solubility of the acetic esters and ketones. Acetic esters and ketones do dissolve anthranilic acid efficiently, but get into the aqueous phase in non-negligible proportions because of their comparatively high water solubility. This firstly results in yield losses and secondly increases complexity in the cleaning of wastewater. The comparatively high water solubility of the acetic esters and ketones makes employment of the extraction process described in WO 2007/088346 A1 in the recovery of anthranilic acid produced by fermentation particularly problematic because the streams of water and hence the absolute yield losses, and also the complexity in the cleaning of wastewater, are considerably greater therein by comparison with a production process based on petrochemical raw materials. Toluene, which is likewise disclosed as a suitable solvent in WO 2007/088346 A1, does not have the disadvantages described, but dissolves anthranilic acid considerably less efficiently, which makes it difficult to develop a practicable process on an industrial scale with toluene as extractant.

CN 104 016 871 A describes the extraction of anthranilic acid-containing wastewater from the production of methyl anthranilate with a mixture of tributyl phosphate and kerosene. The need to use tributyl phosphate leads to an undesirable increase in costs.

CN 104 926 673 A describes the extraction of para-aminobenzoic acid-containing aqueous solutions with ionic liquids. lonic liquids are costly, and so they necessarily have to be recycled for economic use on an industrial scale. By contrast with low molecular weight organic solvents as extractants, however, they cannot simply be purified by a distillation, which makes recycling difficult.

CN 109 850 976 A describes the extraction of para-aminobenzoic acid-containing aqueous solutions with a mixture of tributyl phosphate, 1-octanol and kerosene. The use of such a complex extractant increases costs and makes recycling thereof difficult.

International patent application WO 2015/124686 A1 describes the extraction of aniline that has been obtained by decarboxylation of an ammonium and anthranilate solution with 1-dodecanol. The anthranilic acid can be produced by fermentation or by petrochemical means.

None of the above-described extraction methods is therefore truly satisfactory for the recovery of aminobenzoic acid from a mother liquor obtained in the production of aminobenzoic acid by a fermentative (=biotechnological) route with isolation of the aminobenzoic acid formed by crystallization. This is especially true in the case of production on an industrial scale.

There was thus a need for further improvements in the field of production of aminobenzoic acid by fermentation processes comprising a crystallization step. In particular, it would be desirable to be able to isolate the aminobenzoic acid as completely as possible. For this purpose, it is indispensable to recover the dissolved fraction of aminobenzoic acid remaining in the mother liquor from the crystallization from said mother liquor at least in high proportions. More particularly, it would also be desirable to deplete the mother liquor of organic impurities in the course of such a workup, in order to simplify the workup/disposal of this (considerable) aqueous stream. In this connection, minimum water solubility of the extractant itself is also important. Finally, it would be especially desirable to be able to discharge such organic impurities removed from the mother liquor from the process in an efficient manner.

Taking account of this requirement, the present invention provides a process for recovering aminobenzoic acid from an aqueous mother liquor containing dissolved aminobenzoic acid which is obtained in a crystallization of aminobenzoic acid, wherein the process comprises the following steps:
(A) fermenting a raw material containing a fermentable carbon-containing compound and a nitrogen-containing compound in the presence of microorganisms, in particular at a pH in the range from 3.0 to 11, to form a fermentation process product containing aminobenzoate anions (H₂NC₆H₄COO⁻) and/or aminobenzoic acid (H₂NC₆H₄COOH or H₃N⁺C₆H₄COO⁻);
(B)
   (I) crystallizing aminobenzoic acid during the fermentation by establishing a pH in the fermentation in the range from 3.0 to 4.7, preferably 3.2 to 3.7, more preferably 3.4 to 3.6, most preferably 3.5, followed by removal of the aminobenzoic acid precipitated in the crystallization by filtration, sedimentation or centrifugation to obtain the aqueous mother liquor containing dissolved aminobenzoic acid,
   (II) (1) partially crystallizing aminobenzoic acid during the fermentation, followed by removal of the aminobenzoic acid precipitated in the partial crystallization by filtration, sedimentation or centrifugation to obtain a solution, and (2) crystallizing aminobenzoic acid out of the solution by establishing a pH in the range from 3.0 to 4.7, preferably 3.2 to 3.7, more preferably 3.4 to 3.6, most preferably 3.5, followed by removal of the aminobenzoic acid precipitated in the crystallization by filtration, sedimentation or centrifugation to obtain the aqueous mother liquor containing dissolved aminobenzoic acid,
   or
   (III) crystallizing aminobenzoic acid after the fermentation by establishing a pH in the fermentation process product, optionally (and preferably) after it has been freed of microorganisms, in the range from 3.0 to 4.7, preferably 3.2 to 3.7, more preferably 3.4 to 3.6, most preferably 3.5, followed by removal of aminobenzoic acid precipitated in the crystallization by filtration, sedimentation or centrifugation to obtain the aqueous mother liquor containing dissolved aminobenzoic acid;
(C) extracting the aqueous mother liquor containing dissolved aminobenzoic acid with an alkanol having 8 to 12 carbon atoms, preferably 9 to 11 carbon atoms, to obtain a first alcoholic phase containing aminobenzoic acid and a first aqueous phase;
(D) re-extracting aminobenzoic acid from the first alcoholic phase with (I) an aqueous base solution or (II) an aqueous acid solution to obtain a second aqueous phase containing (I) anions or (II) cations of aminobenzoic acid and a second alcoholic phase; and
(E) crystallizing aminobenzoic acid out of the second aqueous phase
   (I) by guiding the second aqueous phase into the crystallization from (B)(I), (B)(II)(2) or (B)(III) that was conducted at a pH in the range from 3.0 to 4.7, preferably 3.2 to 3.7, more preferably 3.4 to 3.6, most preferably 3.5,
   and/or (preferably or)
   (II) by establishing a pH in the range from 3.0 to 4.7, preferably 3.2 to 3.7, more preferably 3.4 to 3.6, most preferably 3.5, in a step other than (B)(I), (B)(II)(2) and (B)(III), followed by the removal of the aminobenzoic acid thus precipitated by filtration, sedimentation or centrifugation (leaving a further aqueous mother liquor, which is especially disposed of).

It has been found that, completely surprisingly, the use of C₈-C₁₂-alkanols, especially of C₉-C₁₁-alkanols, as extractant (step (C)) in conjunction with a basic re-extraction (step (D)) constitutes a good compromise between the requirement for maximum extraction of the aminobenzoic acid from the mother liquor (in order to achieve a maximum yield) on the one hand and the requirement to obtain a mother liquor of maximum purity (in order to simplify the wastewater processing/disposal) on the other hand.

All pH values in the context of the present invention relate to the temperature at which the corresponding step (e.g. step (B) or (E.II)) is conducted and can simply be measured with a glass electrode. Steps (B) and (E.II) are especially conducted at ambient temperature.

There will firstly follow a brief summary of various possible embodiments of the invention:

In a first embodiment of the invention, which can be combined with all other embodiments, the alkanol used is a primary alkanol.

In a second embodiment of the invention, which is a particular configuration of the first embodiment, the primary alcohol used is 1-decanol.

In a third embodiment of the invention, which can be combined with all other embodiments, the aqueous base solution used in (D)(I) is sodium hydroxide solution, potassium hydroxide solution, a sodium or potassium hydrogencarbonate solution, a sodium or potassium carbonate solution or a mixture of two or more of the aforementioned compounds, and the aqueous acid solution used in (D)(II) is hydrochloric acid, sulfuric acid, phosphoric acid or a mixture of two or more of the aforementioned compounds.

In a fourth embodiment of the invention, which can be combined with all other embodiments, in the re-extraction in (D), a molar ratio of hydroxide ions (in variant (D)(I)) or hydronium ions (in variant (D)(II)) to aminobenzoic acid of 1.0 to 5.0, preferably 1.0 to 2.0 and more preferably 1.0 to 1.5 is observed.

In a fifth embodiment of the invention, which can be combined with all other embodiments, the pH is established in (B) and/or in (E.II) by adding an acid selected from hydrochloric acid, sulfuric acid or phosphoric acid or by adding a base selected from aqueous ammonia, gaseous ammonia, sodium hydroxide solution, potassium hydroxide solution, a sodium or potassium hydrogencarbonate solution or a sodium or potassium carbonate solution. In variants (B)(II) and in particular (B)(III), it is also possible to use the fermentation process product (which then contains aminobenzoate anions) to establish the pH in E(II).

In a sixth embodiment of the invention, which can be combined with all other embodiments, the extraction in (C) is conducted at a temperature of 20° C. to 90° C., preferably 25° C. to 70° C., more preferably 30° C. to 50° C.

In a seventh embodiment of the invention, which can be combined with all other embodiments, the re-extraction in (D) is conducted at a temperature of 20° C. to 90° C., preferably 25° C. to 70° C., more preferably 30° C. to 50° C.

In an eighth embodiment of the invention, which can be combined with all other embodiments,
the fermentable carbon-containing compound is selected from starch hydrolyzate, sugarcane juice, sugarbeet juice, hydrolyzates of lignocellulosic raw materials or a mixture of two or more of the aforementioned carbon-containing compounds,
and
the nitrogen-containing compound is selected from ammonia gas, aqueous ammonia, (at least) one ammonium salt, soya protein, urea or a mixture of two or more of the aforementioned nitrogen-containing compounds.

In a ninth embodiment of the invention, which can be combined with all other embodiments, the microorganisms are selected from Escherichia coli, Pseudomonas putida, Corynebacterium glutamicum, Ashbya gossypii, Pichia pastoris, Hansenula polymorpha, Kluyveromyces marxianus, Yarrowia lipolytica, Zygosaccharomyces bailii or Saccharomyces cerevisiae.

In a tenth embodiment of the invention, which can be combined with all other embodiments, the second alcoholic phase, optionally after a purification, is recycled into the extraction of (C).

In an eleventh embodiment of the invention, which can be combined with all other embodiments,
(F) the first aqueous phase is separated by distillation into a third aqueous phase and a third alcoholic phase, where the third aqueous phase is disposed of as wastewater and the third alcoholic phase is recycled into the extraction of (C).

In a twelfth embodiment of the invention, which can be combined with all other embodiments, (E.II) is conducted.

In a thirteenth embodiment of the invention, which can be combined with all other embodiments, ortho-aminobenzoic acid is produced.

In a fourteenth embodiment of the invention, which can be combined with all other embodiments, para-aminobenzoic acid is produced.

The embodiments briefly outlined above and further possible configurations of the invention are elucidated in detail hereinafter. All the above-described embodiments and the further configurations of the invention described below are mutually and collectively combinable as desired unless the opposite is clearly apparent from the context to a person skilled in the art or is expressly stated.

### Providing the Fermentation Broth that has Especially Been Freed of Microorganisms (=Clarified)

Step (A) of the process of the invention relates to the fermentation of a fermentable carbon-containing compound and a nitrogen-containing compound in the presence of microorganisms to form aminobenzoate anions (H₂NC₆H₄COO⁻) and/or aminobenzoic acid (H₂NC₆H₄COOH or H₃N⁺C₆H₄COO⁻). The fermentation is conducted in a reaction apparatus provided for the purpose, the fermentation reactor. The reaction mixture present in the fermentation reactor is referred to as fermentation broth. The fermentation broth present after fermentation is also referred to in the context of the present invention as fermentation process product.

The fermentation in step (A) is preferably conducted in such a way that the pH in the fermentation broth is in the range from 3.0 to 11, preferably 6.0 to 8.0. If required, the pH can be controlled by addition of aqueous or gaseous ammonia, aqueous potassium hydroxide or aqueous sodium hydroxide (when pH values are too low), or by addition of an aqueous acid, especially of hydrochloric acid, sulfuric acid or nitric acid (when pH values are too high). Different pH ranges within the ranges mentioned may be particularly optimal for different microorganisms; this is elucidated in detail hereinafter.

Preferred microorganisms for the performance of step (I) are prokaryotes (such as bacteria in particular) or eukaryotes (such as yeasts in particular). Particularly preferred here are microorganisms such as Escherichia coli, Pseudomonas putida, Corynebacterium glutamicum, Ashbya gossypii, Pichia pastoris, Hansenula polymorpha, Kluyveromyces marxianus, Yarrowia lipolytica, Zygosaccharomyces bailii or Saccharomyces cerevisiae, particular preference being given to the sole use of Corynebacterium glutamicum, in particular Corynebacterium glutamicum ATCC 13032.

The pH to be maintained in the fermentation, as already mentioned, is guided by the microorganism used. Microorganisms such as Corynebacterium glutamicum, Pseudomonas putida or Escherichia coli are preferably cultured at “neutral to basic pH values” (i.e. in particular at a pH in the range from 6.0 to 11, preferably 6.0 to 8.0). Microorganisms such as Saccharomyces cerevisiae, by contrast, are preferably cultured in an acidic medium (i.e. in particular at a pH in the range from 3.0 to <6.0, preferably 4.0 to <6.0). Depending on the specific pH within the range from 3.0 to 11, the aminobenzoic acid in the fermentation broth is in anionic form (H₂NC₆H₄COO⁻) or in electrically neutral form (H₂NC₆H₄COOH or H₃N⁺C₆H₄COO⁻) (the formation of cations H₃N⁺C₆H₄COOH at pH values in the lowermost part of the range mentioned, i.e. at pH 3.0 or slightly above, cannot be ruled out entirely, but at best accounts for a minor proportion of the aminobenzoic acid present overall):

At pH values in the region of 4.7 or less in particular, preferably of 3.7 or less, more preferably of 3.6 or less, the aminobenzoic acid is predominantly to completely in electrically neutral form and therefore crystallizes spontaneously during the fermentation, and so a separate crystallization step is dispensable and the crystallized aminobenzoic acid can be isolated directly from the fermentation broth (variant (I) of step (B)). At pH values of >4.7 in particular, preferably of 6.0 or more, more preferably of 8.0 or more, the aminobenzoic acid is predominantly to entirely in anionic form, and so a separate crystallization step—variant (III) of step (b); see below for details—is conducted. It is likewise conceivable—variant (II) of step (B)—to conduct the fermentation at such pH values that aminobenzoic acid already precipitates during the fermentation, but the proportion of anionically dissolved aminobenzoic acid can be reduced further by an additional pH adjustment conducted after the fermentation. Variant (III)—performance of the fermentation at such pH values that aminobenzoic acid remains predominantly to completely dissolved in anionic form during the fermentation and the crystallization is conducted in a separate step-is the most preferred among the three variants.

In a preferred configuration of the invention, prokaryotes, especially bacteria, are used as microorganisms. Particular reference is made here to patent applications WO 2015/124686 A1 and WO 2015/124687 A1, which describe fermentation processes with use of bacteria (see, for example, WO 2015/124687 A1, (i) page 15 line 8 to page 16 line 30, (ii) example 1 (page 29 lines 4 to 26), (iii) example 3 (in particular page 34 lines 10 to 18), (iv) example 4 (in particular page 55 lines 9 to 31) and which afford the anion of aminobenzoic acid (aminobenzoate anion) as the direct fermentation process product, i.e. are suitable for variant (III). In particular, bacteria used are those that are capable of converting a fermentable carbon-containing compound to aminobenzoic acid in the presence of a suitable nitrogen source without the aminobenzoic acid thus formed being consumed straightaway in intracellular biochemical processes, with the result that aminobenzoic acid is enriched in the cell and is ultimately transferred into the fermentation broth.

In another preferred configuration of the invention, eukaryotes, especially yeasts, are used as microorganisms. In this connection, reference is made in particular to international patent application WO 2017/102853 A1. In particular, yeast cells capable of converting a fermentable carbon-containing compound to aminobenzoic acid in the presence of a suitable nitrogen source without the aminobenzoic acid thus formed being consumed straightaway in intracellular biochemical processes are used, with the result that aminobenzoic acid is enriched in the cell and is ultimately transferred into the fermentation broth. Yeasts are preferably cultivated in an acidic medium and are therefore suitable for variants (I) and (II).

Two routes are available in principle for obtaining prokaryotes of this kind or eukaryotes of this kind, and these can also be combined in a preferred configuration:
(i) The enzymatic reactions in the aminobenzoic acid metabolic pathway of the prokaryotic or eukaryotic cell can be increased such that aminobenzoic acid is produced more rapidly than it is consumed.
(ii) The conversion reactions which convert aminobenzoic acid to further metabolites or products (e.g. tryptophan) can be reduced or switched off, with the result that even the rate of aminobenzoic acid formation in wild-type strains is sufficient to lead to an enrichment of aminobenzoic acid in the cell.

Methods of obtaining prokaryotic or eukaryotic organisms with the aforementioned properties are known from the prior art. Suitable prokaryotes or eukaryotes can be identified, for example, by screening for mutants which secrete aminobenzoic acid into the surrounding medium. However, preference is given to the specific modification of key enzymes by means of genetic engineering methods. Using customary genetic engineering methods, gene expression and enzyme activity can be enhanced, reduced or even completely suppressed. Recombinant strains are the result. For the particularly preferred ortho isomer, a preferred embodiment is described hereinafter; application to the other isomers is within the customary ability of a person skilled in the art:

More preferably, the prokaryotes or eukaryotes capable of converting a fermentable carbon-containing compound to aminobenzoic acid in the presence of a nitrogen-containing compound contain to a modification the anthranilate phosphoribosyltransferase activity, which lowers said enzyme activity. As a result of said modification, the conversion of ortho-aminobenzoate to N-(5-phospho-D-ribosyl)anthranilate is reduced or completely suppressed. This causes enrichment of aminobenzoic acid in the cell. The expression “anthranilate phosphoribosyltransferase activity” refers here to an enzyme activity which catalyzes the conversion of ortho-aminobenzoate to N-(5-phospho-D-ribosyl)anthranilate.

In yeasts, anthranilate phosphoribosyltransferase activity is genetically encoded by the native gene TRP4 (YDR354W). In the bacterium Corynebacterium glutamicum, anthranilate phosphoribosyltransferase activity is encoded by the trpD gene (cg3361, Cgl3032, NCgl2929). In the case of Pseudomonas putida, the encoding is effected via the trpD gene (PP_0421) within the trpDC operon.

The described lowering of anthranilate phosphoribosyltransferase activity can be achieved in principle in three ways:
(i) The regulation of the expression of the gene for anthranilate phosphoribosyltransferase activity can be modified such that the transcription of the gene or subsequent translation is reduced or suppressed.
(ii) The nucleic acid sequence of the gene for anthranilate phosphoribosyltransferase activity can be modified such that the enzyme which is encoded by the modified gene has a lower specific activity.
(iii) The native gene for anthranilate phosphoribosyltransferase activity can be replaced with a different gene which originates from a different organism and encodes an enzyme having a specific anthranilate phosphoribosyltransferase activity lower than that of the native genes mentioned above (e.g. TRP4, trpD or trpDC).

Aminobenzoic acid occurs in three isomeric forms (ortho-, meta- and para-aminobenzoic acid). In principle, the process according to the invention can be applied to all three isomers, either in isomerically pure form or as mixtures of different isomers. However, preference is given to the production of ortho-aminobenzoic acid or para-aminobenzoic acid, especially in isomerically pure form. Particular preference is given to the production of ortho-aminobenzoic acid, especially in isomerically pure form. What is meant by “isomerically pure” in this connection, in the terminology of the present invention, is that the molar proportion of the desired aminobenzoic acid isomer, based on all the aminobenzoic acid isomers present, is at least 99.0 mol %, preferably at least 99.9 mol %, more preferably 100 mol %. As known in the specialist field, the formation of the desired isomer can be controlled enzymatically. For instance, by the shikimate pathway, chorismate can be converted enzymatically to anthranilate (=anion of ortho-aminobenzoic acid). Alternatively, there are also enzyme-catalysed reactions of chorismate to give para-aminobenzoate (=anion of para-aminobenzoic acid).

Irrespective of which microorganism is used and which isomer is desired, the fermentation broth at the start of the fermentation in step (A) comprises recombinant cells of the microorganism used and at least one fermentable carbon-containing compound (and at least one nitrogen-containing compound as nitrogen source). Preferably, the fermentation broth additionally contains further constituents selected from the group consisting of buffer systems, inorganic nutrients, amino acids, vitamins and further organic compounds which are required for the growth or housekeeping metabolism of the recombinant cells. The fermentation broth is water-based. After the fermentation process has been started, the fermentation broth also comprises aminobenzoic acid, the target fermentation product.

As already mentioned, a fermentable carbon-containing compound in the context of the present invention is understood to mean any organic compound or mixture of organic compounds that can be used to produce aminobenzoic acid by the recombinant cells of the microorganism used. The production of aminobenzoic acid can take place here in the presence or in the absence of oxygen.

Preference is given here to those fermentable carbon-containing compounds which can additionally serve as energy and carbon source for the growth of the recombinant cells of the microorganism used. Particularly suitable are starch hydrolyzate, sugarcane juice, sugarbeet juice and/or hydrolyzates of lignocellulose-containing raw materials. The nitrogen source used is preferably ammonia gas, aqueous ammonia, (at least) one ammonium salt, soya protein and/or urea.

In one embodiment of the invention, step (A) is performed continuously, i.e. the reactants are fed continuously to the fermentation reactor and the product is withdrawn continuously from the fermentation reactor. The product withdrawn continuously from the fermentation reactor in the simplest case is the fermentation broth including the microorganisms present therein. However, it is also conceivable to use known separation methods (especially filtration) in order to retain the microorganisms in the fermentation reactor and to withdraw a clarified fermentation broth therefrom. Such a clarification of the fermentation broth can of course also be undertaken outside the fermentation reactor, especially by filtration, centrifugation or sedimentation. In variant (III), preference is given to clarification of the fermentation broth before the crystallization in step (B) (see further down for details).

In another embodiment of the invention, step (A) is conducted in a discontinuous process regime (“batchwise mode”) in fermentation cycles. A fermentation cycle preferably comprises the initial charging or addition of microorganisms to a culture medium, the initial charging and/or addition of nutrients, the buildup of microorganisms, the formation of the desired product, i.e. the aminobenzoic acid, and the complete or partial emptying of the reactor on conclusion of the fermentation. In one variant of the batchwise mode of operation (called “fed-batch mode”), the reactants are fed to the fermentation reactor (continuously or discontinuously [i.e. in portions]) for as long as the reactor volume allows it without products—possibly excluding gaseous constituents that are discharged to an offgas system via a fermentation reactor connection—being withdrawn from the fermentation reactor. The reaction is stopped after addition of the maximum possible amount of reactants and the product mixture is withdrawn from the fermentation reactor. Even in the case of a discontinuous process regime, preference is given to clarification of the fermentation broth, especially by filtration, centrifugation or sedimentation, prior to the crystallization in step (B) according to variant (III).

Irrespective of the exact mode of operation, the fermentation reactor preferably comprises devices for measuring important process parameters such as temperature, pH, concentration of substrate and product, dissolved oxygen content, and cell density of the fermentation broth. In particular, the fermentation reactor preferably comprises devices for adjusting at least one (preferably all) of the aforementioned process parameters.

Suitable fermentation reactors are stirred tanks, membrane reactors, plug flow reactors or loop reactors. Particularly preferred for both aerobic and anaerobic fermentations are stirred tank reactors and loop reactors (preferably airlift reactors in which circulation of the liquid in the reactor is achieved by sparging).

### Crystallization of the Aminobenzoic Acid from the Fermentation Broth that has Especially been Clarified

In step (B) of the process according to the invention, the aminobenzoic acid is crystallized. This is accomplished in variant (I) (exclusively) in the fermentation reactor itself, i.e. still during the fermentation in step (A). The precipitated aminobenzoic acid, which is obtained in a mixture with the microorganisms, is separated off by filtration, sedimentation or centrifugation after the fermentation has ended. The procedure for separation of the two may, for example, be such that the aminobenzoic acid is selectively dissolved in maximum concentration (for example with concentrated base solution or with an organic solvent) and then separated out of this solution again if required (by another crystallization or concentration). An example of a suitable organic solvent is aniline. This is also advantageous especially when the aminobenzoic acid obtained is to be decarboxylated to aniline because no solvent extraneous to the system is then introduced. Moreover, the solution of aminobenzoic acid can be directly decarboxylated to aniline, in which case the aniline actually displays a catalytic effect. The procedure in the workup of the fermentation process product according to variant (II) may be as described here for variant (I).

In variants (II) and (III), as well as the fermentation reactor, an additional industrial apparatus suitable for crystallisations is used, known in the specialist field as crystallizer. The crystallization in such a crystallizer can be conducted in the same way for variants (II) and (III) and is therefore described collectively hereinafter for both variants:

Suitable crystallizers are, for example, stirred tanks or forced circulation crystallizers, such as those of the “Oslo type”. In the crystallizer, the pH is adjusted to a value in the range from 3.0 to 4.7, preferably 3.2 to 3.7, more preferably 3.4 to 3.6 and most preferably 3.5. This is preferably accomplished by adding an acid selected from hydrochloric acid, sulfuric acid or phosphoric acid. This adjustment of pH results in conversion of the aminobenzoic acid anions (H₂NC₆H₄COO⁻) predominantly to completely to the electrically neutral form (H₂NC₆H₄COOH or H₃N⁺C₆H₄COO⁻) and crystallization thereof. This type of crystallization is also referred to as reactive crystallization. The crystallized aminobenzoic acid can be isolated by filtration, sedimentation or centrifugation, leaving the aqueous mother liquor containing dissolved aminobenzoic acid.

It has been found to be useful to feed the fermentation broth and the acid to the crystallizer by means of spatially separate feed devices (that are as far apart as possible). This achieves the effect that the reactants are very well mixed with the reactor contents before the acid-base reaction occurs. Examples of useful feed devices include pipelines, preferably with barrier valves. In one embodiment, the feed device for the fermentation broth and the feed device for the acid are disposed at opposite sites on the reactor wall, (essentially) at right angles thereto. In another embodiment, the feed device for the fermentation broth and the feed device for the acid are arranged (essentially) parallel to the reactor wall, where the feed devices are opposite one another and as close as possible to one another on the reactor wall, especially directly opposite one another.

It is possible to divide the crystallizer used in variants (II) and (III) of step (B) into chambers by means of suitable internals. It is possible to adjust the flow direction by choice of stirrer geometry and mode of operation of the stirrer. It is likewise possible to provide the crystallizer with an external pump circulation system, in which case one of the two reactants—fermentation broth or acid—is introduced into the pump circulation system and the other directly into the crystallizer. If a crystallizer is operated with sifter and pumped circulation system, the pumped circulation system is used at the sifter base for agitation or at the side of the sifter.

The crystallization in the crystallizer can be performed continuously or batchwise. Continuous performance is preferred. Irrespective of the mode of operation (continuous or batchwise), the exact operating parameters (inter alia) are determined by the desired crystal size, which can be adjusted via the dwell time/reaction time and the level of oversaturation (large crystal sizes are promoted by long dwell times/long reaction times and low levels of oversaturation).

The crystallization is preferably conducted in the presence of seed crystals:

The preferred procedure in a crystallization conducted batchwise is first to charge the crystallizer with the fermentation broth and to adjust it to a defined temperature (preferably 5° C. to 40° C., for example 20° C.). If the pH of the fermentation broth is significantly above the solubility limit of aminobenzoic acid at the chosen temperature, the fermentation broth is first slightly acidified before the actual addition of acid (preferably with one of the acids already mentioned [hydrochloric acid, sulfuric acid or phosphoric acid], more preferably with hydrochloric acid, especially with 37% hydrochloric acid), and to a pH corresponding to or at least close to the solubility limit of aminobenzoic acid at the chosen temperature (preferably pH 5.2 to 5.8, especially pH 5.5). This slight acidification can be effected rapidly. Then seed crystals of the desired polymorph of aminobenzoic acid are added, preferably polymorph (form) I. This polymorph has comparatively low solubility and therefore promotes very substantial recovery of the aminobenzoic acid. The amount of the seed crystals added is preferably about 0.1% to 1% of the aminobenzoic acid dissolved in the fermentation broth. In this way, a suspension of seed crystals is obtained (see also WO 2017/085170 A1). Subsequently, the pH is adjusted by addition of acid (in the case of the preceding slight acidification, the same acid as used therein) to 3.0 to 4.7, preferably 3.2 to 3.7, more preferably 3.4 to 3.6, most preferably 3.5. The acid is preferably added gradually; for example within 1 h with 1 kg of initially charged fermentation broth and use of 37% hydrochloric acid. After the addition of acid has ended, stirring is continued for a certain time, especially for the same period of time taken for the addition of the acid after addition of the seed crystals. The precipitated aminobenzoic acid is then separated off by filtration, sedimentation or centrifugation (preferably by vacuum filtration) and preferably more than once (especially twice) with an aqueous acidic wash liquid (especially the same acid that was used for precipitation) at a pH of 3.0 to 4.7, preferably 3.2 to 3.7, more preferably 3.4 to 3.6, most preferably 3.5.

In the case of a crystallization performed continuously, seed crystals generally only have to be added specially on startup of the continuous process since further seed crystals form later on in situ of their own accord (called secondary nucleation). The suspension of seed crystals required for startup can be provided here as described above for batchwise crystallization. The workup (removal and washing of the crystallized aminobenzoic acid) can likewise be effected as described above.

Variants (I) and (II) in the case of crystallization in a crystallizer differ only in one aspect, namely in that the starting material to be treated with acid, in the case of variant (II), is a solution (namely the solution obtained after the aminobenzoic acid that has already precipitated during the fermentation has been separated off; this separation likewise removes suspended microorganisms), and, in the case of variant (III), a suspension of the microorganisms in the fermentation process product. In the case of variant (III), it is therefore preferable, before the crystallization is performed, to free the fermentation process product of the microorganisms suspended therein (to clarify it) by filtration, centrifugation or sedimentation. If this is not done, when the aminobenzoic acid precipitated in the crystallization is separated off, it will be obtained in a mixture with microorganisms. The procedure for separation of the two may be as described further up for variant (I) (selective dissolution of the aminobenzoic acid and, if required, separation of the aminobenzoic acid again from the solution obtained). Since this is complex, however, and yield losses cannot be ruled out, preference is given to the removal of the microorganisms before the crystallization.

As well as the preferentially conducted clarification, the fermentation broth from step (A) can be subjected to further pre-treatment steps before being fed to step (B). Particular mention should be made here of a decolorization of the fermentation broth (having especially been clarified). Such a decolorization is preferably conducted in such a way that the fermentation broth, or fermentation broth freed of microorganisms, is passed through a column with solid packing in order to remove dyes by adsorption. A possible solid phase that can be used is, for example, kieselguhr or an ion-exchange packing. Such a decolorization is preferably conducted when colored substances that could disrupt the subsequent crystallization in step (B) are present in the fermentation broth. In variant (II), the solution obtained after the aminobenzoic acid already crystallized in the fermentation has been separated off may likewise be subjected to such a decolorization before the crystallization in (B) (III).

### Extraction of the Aqueous Mother Liquor Containing Dissolved Aminobenzoic Acid with A C₈-C₁₂, Especially C₉-C₁₁, Alkanol

In step (C) of the process according to the invention, the mother liquor obtained in step (B), containing dissolved benzoic acid, is extracted with an alkanol having 8 to 12, preferably 9 to 11, carbon atoms. After phase separation, this affords a first alcoholic phase containing aminobenzoic acid and a first aqueous phase. Preference is given to using primary alkanols, more preferably 1-decanol, as extractant. The extraction can be conducted at temperatures of 20° C. to 90° C., preferably 25° C. to 70° C., more preferably 30° C. to 50° C., especially also at ambient temperature. Suitable apparatuses for the performance of the extraction are, for example, what are called mixer-settlers or extraction columns having a number of theoretical plates of preferably 3 to 10, more preferably 3 to 7, most preferably 4 to 6. The mass-based phase ratio of the organic phase to the aqueous phase is preferably 0.20 to 1.0, more preferably 0.20 to 0.50, most preferably 0.35 to 0.50.

The extraction leaches not only aminobenzoic acid out of the mother liquor, but also organic impurities. This has the advantage that the extracted mother liquor (=the first aqueous phase) is in comparatively pure form, which facilitates the further purification thereof (see step (F) further down).

### Re-Extraction of Aminobenzoic Acid from the First Alcoholic Phase

In step (D) of the process of the invention, the first alcoholic phase obtained in step (B) is extracted with an aqueous base solution (I) or acid solution (II) in order to recover the aminobenzoic acid again from this alcoholic phase (called re-extraction). In the treatment with (I) base or (II) acid, the water solubility of the aminobenzoic acid is increased by conversion to a negatively charged form (by deprotonation with a base) or positively charged form (by protonation with an acid) overall, such that the resultant anions or cations are transferred predominantly to completely to the aqueous phase. In this case, therefore, after phase separation, a second aqueous phase containing in case (I) anions (H₂NC₆H₄COO⁻) or in case (II) cations (H₃N⁺C₆H₄COOH) of the aminobenzoic acid and a second alcoholic phase are obtained. Since comparatively polar organic impurities are obtained in the fermentation (see the examples) and in particular these comparatively polar impurities are transferred at least in portions to the second aqueous phase in this step, it is preferable to take measures for discharge of such impurities (in this regard see the description of step (E) further down). Just like step (B), this extraction can also be conducted at temperatures of 20° C. to 90° C., preferably 25° C. to 70° C., more preferably 30° C. to 50° C., especially also at ambient temperature.

The aqueous base solution used for step (D)(I) is preferably sodium hydroxide solution, potassium hydroxide solution, a sodium or potassium hydrogencarbonate solution, a sodium or potassium carbonate solution or (less preferably) a mixture of two or more of the aforementioned compounds. Sodium hydroxide solution and potassium hydroxide solution are particularly preferred. Irrespective of the base used, it is preferable to observe a molar ratio of hydroxide ions to aminobenzoic acid of 1.0 to 5.0, preferably 1.0 to 2.0 and more preferably 1.0 to 1.5. The aqueous acid solution used for step (D)(II) is preferably hydrochloric acid, sulfuric acid, phosphoric acid or (less preferably) a mixture of two or more of the aforementioned compounds. Preference is given to hydrochloric acid or sulfuric acid. Irrespective of the acid used, it is preferable to observe a molar ratio of hydronium ions (H₃O⁺ ions) to aminobenzoic acid of 1.0 to 5.0, preferably 1.0 to 2.0 and more preferably 1.0 to 1.5.

Depending on the procedure chosen, the second aqueous phase is thus basic (pH in particular 7.0 or less, preferably 7.0 to 14, more preferably 7.0 to 13; (D)(I)) or acidic (pH in particular 2.0 or less, preferably 0.0 to 2.0, more preferably 0.0 to 1.0; (D)(II))).

Suitable apparatus for this re-extraction is the same as described above for step (C). The mass-based phase ratio of the organic phase to the aqueous phase is preferably 0.20 to 1.0, more preferably 0.20 to 0.50, most preferably 0.20 to 0.40.

The second alcoholic phase obtained after phase separation, optionally after a purification for removal of impurities that have got into this phase in the phase separation, is preferably recycled into the extraction of step (C), where it is used as a constituent (possibly even as the sole constituent) of the C₈-C₁₂, especially C₉-C₁₁, alkanol used as extractant. A purification performed optionally especially comprises a distillation.

### Crystallization of Aminobenzoic Acid Out of the Second Aqueous Phase

In the subsequent step (E) of the process of the invention, the aminobenzoic acid dissolved in the second aqueous phase is crystallized. For this purpose, the pH of the second aqueous phase has to be adjusted to a value in the range from 3.0 to 4.7, preferably 3.2 to 3.7, more preferably 3.4 to 3.6, most preferably 3.5, which is accomplished by reducing the pH (in the case of basic re-extraction in (D)(I)) or increasing the pH (in the case of acidic re-extraction in (D)(II).

In the simplest case, this can be effected by—(E)(I)—recycling the second aqueous phase into the crystallization from step (B), namely into a crystallization as per (B)(I), (B)(II)(2) or (B)(III):

If the second aqueous phase, for crystallization, is directed into the fermentation (i.e. into step (B)(I)), the pH that results after mixing of fermentation broth and second aqueous phase must be within a range that results in the crystallization of the aminobenzoic acid (see the description of steps (A) and (B) further up for details). Whether further acid or base has to be added at all and, if so, to what extent for this purpose in addition to the second aqueous phase is decided depending on the circumstances of the individual case and will be immediately apparent to the person skilled in the art. Preferably, in the case of recycling of the second aqueous phase into the crystallization in (B)(I), the re-extraction is conducted under basic conditions (i.e. as per (D)(I)). The dosage of the second aqueous phase, which is then basic, into the fermentation broth-which is acidic in this variant-is undertaken in such a way that the resulting pH is within the desired range from 3.0 to 4.7, preferably 3.2 to 3.7, more preferably 3.4 to 3.6, most preferably 3.5.

If the second aqueous phase, for crystallization, is directed into a crystallizer other than the fermentation reactor (i.e. into step (B)(II)(2) or (B)(III)), the pH that results after mixing of the solution from (B)(II)(1) or of the fermentation process product and second aqueous phase must be within a range that enables the crystallization of aminobenzoic acid, i.e. in the range from 3.0 to 4.7, preferably 3.2 to 3.7, more preferably 3.4 to 3.6, most preferably 3.5. In variants (B)(II) and (B)(III), the pH at this point (i.e. in the solution from (B)(1) or in the fermentation process product) is comparatively high, such that aminobenzoate anions are present. The pH of the solution from (B)(1) or of the fermentation process product thus has to be reduced. In the case of performance of the re-extraction under acidic conditions as per (D)(II), the requirement for acid for this purpose is partly covered by the second aqueous phase itself. In the case of performance of the re-extraction under basic conditions as per (D)(I), the requirement for acid is increased correspondingly by addition of the second aqueous phase.

In the case of performance of step (E) as per (E)(I), the aminobenzoic acid thus crystallizes out of the second aqueous phase together with the aminobenzoic acid from the fermentation broth, and is also processed together therewith as described (see FURTHER PROCESSING OF THE AMINOBENZOIC ACID). In this variant, organic impurities dissolved in the second aqueous phase are preferably discharged via purge streams.

However, it is also possible—(E)(II)—to direct the second aqueous phase, in a step different than (B)(I), (B)(II)(2) and (B)(III), into a (comparatively small) crystallizer (other than the crystallizer used in step (B)(II)(2) and (B)(III)) and to crystallize aminobenzoic acid therein:

For this purpose, the pH has to be adjusted to a value in the range from 3.0 to 4.7, preferably 3.2 to 3.7, more preferably 3.4 to 3.6, most preferably 3.5. The pH is preferably adjusted
(i)—(in the case of performance of the re-extraction as per (D)(I))—by adding an acid selected from hydrochloric acid, sulfuric acid or phosphoric acid
or
(ii)—(in the case of performance of the extraction as per (D)(II)—by adding a base selected from aqueous ammonia, gaseous ammonia, sodium hydroxide solution, potassium hydroxide solution, a sodium or potassium hydrogencarbonate solution or a sodium or potassium carbonate solution, or else by adding a portion of the fermentation process product. The latter naturally requires the pH of the fermentation process product to be significantly above the pH of the second aqueous phase and also high enough to bring the resulting mixture to a pH in the range from 3.0 to 4.7, preferably 3.2 to 3.7, more preferably 3.4 to 3.6, most preferably 3.5. Therefore, the use of a portion of the fermentation process product to increase the pH of the second aqueous phase in the case of acidic re-extraction is restricted to variants (B)(II) and (B)(III) and is suitable for variant (B)(III) in particular.

Phase separation by filtration, sedimentation or centrifugation leaves a further aqueous mother liquor in the embodiment according to (E)(II). This further aqueous mother liquor then contains the organic impurities extracted in step (C) from the mother liquor obtained in step (B) at least in a considerable proportion. Since the further aqueous mother liquor from step (D) is obtained in a much smaller amount (based on the phase ratios of the two upstream extraction steps) than the first aqueous phase (i.e. the organic impurities are concentrated), the further aqueous mother liquor then forms a comparatively small aqueous phase that still contains the aminobenzoic acid product of value in a comparatively negligible concentration at best, but is comparatively rich in organic impurities and can therefore be disposed of. In this way, secondary components are discharged from the process in a simple manner, and so they cannot be accumulated to such a significant degree. The obtaining of the further aqueous mother liquor in a comparatively small amount also facilitates disposal (either by purification, for example by precipitation of the organic impurities, followed by feeding into the wastewater or—albeit less preferably—by incineration after evaporative concentration). This embodiment offers a simple outlet for organic impurities from the process and is therefore preferred.

It is likewise possible to combine the aforementioned embodiments (E)(I) and (E)(II) in such a way that a first portion of the second aqueous phase according to (E)(I) and a second portion of the second aqueous phase according to (E)(II) are subjected to further treatment. However, preference is given to conducting just one of variants (E)(I) and (E)(II). This is true especially when the fermentation is conducted at acidic pH values of 3.0 to 4.7 and, as a result, the aminobenzoic acid in variant (B)(I) spontaneously crystallizes during fermentation. In this case, it is preferable to treat the second aqueous phase only according to variant (E)(II). This avoids any need to additionally add acid to the fermentation in order to keep the pH in the range from 3.0 to 4.7.

### Workup of the First Aqueous Phase

The first aqueous phase obtained in step (C) is preferably subjected to further workup in a step (F). Since this first aqueous phase in the process of the invention is obtained in comparatively pure form, a simple distillation is sufficient for the purpose of removing the (small) proportions of alkanol dissolved in the first aqueous phase. In this case, a third alcoholic phase is distilled off, which is recycled into the extraction of step (C), where it is used as a constituent of the C₈-C₁₂, especially C₉-C₁₁, alkanol to be used as extractant. The distillation leaves a third aqueous phase, which is disposed of as wastewater. Since, in the process of the invention, the third aqueous phase contains organic impurities in comparatively small proportions at most, it can generally be (and preferably indeed is) sent directly to a water treatment plant, especially also a biological water treatment plant.

### Further Workup of the Aminobenzoic Acid

The aminobenzoic acid obtained in step (C) and/or in step (E.II) can optionally be subjected to further purification. In the case of performance of step (E.II), this is preferably done after purification with the aminobenzoic acid obtained in step (B). Such a purification is known per se from the prior art (see, in particular, WO 2015/124687 A1 and especially WO 2015/124687 A1, page 18, line 4 to page 18, line 6) and is preferably carried out by one or more washes with aqueous wash media, preferably water. In order to avoid yield losses, the pH of the aqueous wash medium is preferably adjusted to a value in the range from 3.0 to <4.0, preferably to a value of 3.5 to 3.7.

The optionally purified aminobenzoic acid can optionally be sent, in a step (G), to a conversion to an aminobenzoic acid conversion product, i.e. to a product which is obtained by a further chemical conversion of the aminobenzoic acid. In the case of performance of step (E.II), this is also preferably done after purification with the aminobenzoic acid obtained in step (B). Selected further conversions of the aminobenzoic acid obtained are:
(I) decarboxylation of the aminobenzoic acid to aniline;
(II) decarboxylation of the aminobenzoic acid to aniline, followed by acid-catalyzed reaction of the aniline with formaldehyde to form di- and polyamines of the diphenylmethane series;
(III) decarboxylation of the aminobenzoic acid to aniline, followed by acid-catalyzed reaction of the aniline with formaldehyde to form di- and polyamines of the diphenylmethane series, followed by reaction with phosgene to form di- and polyisocyanates of the diphenylmethane series;
(IV) decarboxylation of the aminobenzoic acid to aniline, followed by the conversion of the aniline to an azo compound;
(V) conversion of the aminobenzoic acid to an amide; and
(VI) conversion of the aminobenzoic acid to a conductive polymer such as preferably polyanthranilic acid.

Such conversions are known in the prior art and therefore do not require any detailed description at this point.

There follows a detailed elucidation of the invention by examples.

### EXAMPLES

### Analysis

For the evaluation of the distribution of a component between two phases, the two phases were analyzed by HPLC-MS (with identical sample preparation and method). The coefficients of distribution were then determined as the ratio of peak areas for the same retention time.

### Examples 1 to 9: Extraction of Aminobenzoic Acid from a Mother Liquor

A fermentation broth containing anions of ortho-aminobenzoic acid (from step (A)) was clarified by filtration and then subjected to a crystallization with hydrochloric acid at pH=3.5±0.2 (step (B), variant (III)). The remaining mother liquor was extracted in one stage with various solvents (step (C)). The starting concentration of ortho-aminobenzoic acid in the mother liquor was 8.7 g/L. A phase ratio of organic to aqueous phase of 0.5 was used. The extraction was conducted at 30° C. The experiments were assessed by ascertaining the partition coefficients K (=ratio of the ortho-aminobenzoic acid [oAB] concentration in g/L in the organic phase [ORG=first alcoholic phase] to the aqueous phase [AQ=first aqueous phase]), the solubilities of the extractant in the aqueous phase after the extraction (=first aqueous phase) and—in four cases—the proportion by mass of organic secondary components (SC) in the organic extract phase (=first alcoholic phase) based on the totality of the organic secondary components in the two phases (=m(SC)_{ORG}/m(SC)_{TOT}). For this purpose, a separation was conducted by HPLC; organic impurities that elute earlier than ortho-aminobenzoic acid are referred to collectively as polar secondary components, and organic impurities that elute later than ortho-aminobenzoic acid as nonpolar secondary components.

TABLE 1
Partition coefficient, water solubility and secondary
component distribution for various extractants

| | | | Solubility | m(SC)_{ORG}/m(SC)_{TOT} | |
|---|---|---|---|---|---|
| | | | in water | Polar | Nonpolar |
| | | K = c(oAB)_{ORG}/ | at RT | secondary | secondary |
| Ex. | Extractant | c(oAB)_{AQ} | [g/L] | components | components |
| 1 | Kerosene^{[a]} | 0.0 | <0.02 | n.d. | n.d. |
| 2 | Isododecane^{[a]} | 0.0 | insoluble | n.d. | n.d. |
| 3 | Methylcyclopentane^{[a]} | 0.0 | insoluble | n.d. | n.d. |
| 4 | Toluene^{[a]} | 1.29 | 0.5 | 0.00 | 0.29 |
| 5 | Oleyl alcohol^{[a]} | 6.82 | insoluble | n.d. | n.d. |
| 6 | 1-Decanol^{[b]} | 9.01 | 0.04 | 0.09 | 0.65 |
| 7 | 1-Dodecanol^{[b]} | 8.56 | 0.004 | n.d. | n.d. |
| 8 | Butyl acetate^{[a]} | 21.63 | ~6 | 0.08 | 0.62 |
| 9 | 2-Butanone^{[a]} | 26.12 | ~250 | 0.24 | 0.73 |

^{[a]}noninventive extractant
^{[b]}inventive extractant
n. d. = not determined

The extraction of ortho-aminobenzoic acid with hydrocarbons was successful only to a limited degree, if at all. Extraction with oleyl alcohol was better, but still not yet satisfactory. Only extraction with the inventive extractants 1-decanol and 1-tridecanol leads to technically satisfactory results. The extraction of ortho-aminobenzoic acid with very polar solvents such as butyl acetate and 2-butanone is even more successful, but leads to a high concentration of solvents in the aqueous phase. In processes with aminobenzoic acid obtained by fermentation, this is unacceptable because of the very large volumes of aqueous streams compared to chemical processes.

It is found that, with 1-decanol, organic impurities can be extracted similarly effectively to the case of use of more polar solvents. This enables reduction in the proportion of organic impurities in the first aqueous phase and discharge of such impurities as described further up.

### Examples 10 and 11: Basic Re-Extraction (Step (D)(I)) of Ortho-Aminobenzoic Acid from Solutions in Alkanols

Proceeding from these results, re-extraction with an aqueous base solution (sodium hydroxide solution here) was examined. For each of 1-decanol (example 10) and 1-dodecanol (example 11), extraction was performed from various synthetically enriched organic phases. The extraction was conducted with a molar ratio of NaOH to aminobenzoic acid of 1.2. Solutions with an aminobenzoic acid concentration of 15 g/L, 20 g/L and 25 g/L with a mass-based phase ratio of base solution to organic phase of 0.5 were extracted at 30° C. A recovery of aminobenzoic acid of 80%, averaged over all experiments, was found here for one-stage extraction from 1-decanol solutions, and 85% for one-stage extraction from 1-dodecanol solutions.

### Examples 12 and 13: Acidic Re-Extraction (Step (D)(II)) of Ortho-Aminobenzoic Acid from a Solution in 1-Decanol

Proceeding from these results, in addition, re-extraction with an aqueous acid solution (hydrochloric acid or sulfuric acid here) was examined. Solutions of ortho-aminobenzoic acid in 1-decanol (with 1.5% by mass of ortho-aminobenzoic acid) were in each case extracted with aqueous acid solution having a concentration of 1.0 mol/L with a mass-based phase ratio of organic phase to acid solution of 1.0 at 30° C. When hydrochloric acid was used as extractant, 87.4% of the ortho-aminobenzoic acid was recovered in the case of one-stage extraction (example 12a), 98.1% in the case of two-stage extraction (example 12b), and 99.5% in the case of three-stage extraction (example 12c). When sulfuric acid was used as extractant, 90.3% of the ortho-aminobenzoic acid was recovered in the case of one-stage extraction (example 13a), 99.3% in the case of two-stage extraction (example 13b), and 99.3% in the case of three-stage extraction (example 13c).

1. A process for recovering aminobenzoic acid from an aqueous mother liquor containing dissolved aminobenzoic acid which is obtained in a crystallization of aminobenzoic acid, wherein the process comprises:
(A) fermenting a raw material containing a fermentable carbon-containing compound and a nitrogen-containing compound in the presence of microorganisms to form a fermentation process product containing aminobenzoate anions and/or aminobenzoic acid;
(B)
(I) crystallizing aminobenzoic acid during the fermentation by establishing a pH in the fermentation in the range from 3.0 to 4.7, followed by removal of the aminobenzoic acid precipitated in the crystallization by filtration, sedimentation or centrifugation to obtain the aqueous mother liquor containing dissolved aminobenzoic acid,
(II)(1) partially crystallizing aminobenzoic acid during the fermentation, followed by removal of the aminobenzoic acid precipitated in the partial crystallization by filtration, sedimentation or centrifugation to obtain a solution, and (2) crystallizing aminobenzoic acid out of the solution by establishing a pH in the range from 3.0 to 4.7, followed by removal of the aminobenzoic acid precipitated in the crystallization by filtration, sedimentation or centrifugation to obtain the aqueous mother liquor containing dissolved aminobenzoic acid, or
(III) crystallizing aminobenzoic acid after the fermentation by establishing a pH in the fermentation process product in the range from 3.0 to 4.7, followed by removal of aminobenzoic acid precipitated in the crystallization by filtration, sedimentation or centrifugation to obtain the aqueous mother liquor containing dissolved aminobenzoic acid;
(C) extracting the aqueous mother liquor containing dissolved aminobenzoic acid with an alkanol having 8 to 12 carbon atoms to obtain a first alcoholic phase containing aminobenzoic acid and a first aqueous phase;
(D) re-extracting aminobenzoic acid from the first alcoholic phase with (I) an aqueous base solution or (II) an aqueous acid solution to obtain
a second aqueous phase containing (I) anions of aminobenzoic acid or (II) cations of aminobenzoic acid
and
a second alcoholic phase;
and
(E) crystallizing aminobenzoic acid out of the second aqueous phase
(I) by guiding the second aqueous phase into the crystallization from (B)(I), (B)(II)(2) or (B)(III) that was conducted at a pH in the range from 3.0 to 4.7, and/or
(II) by establishing a pH in the range from 3.0 to 4.7, in a step other than (B)(I), (B)(II)(2) and (B)(III), followed by the removal of the aminobenzoic acid thus precipitated by filtration, sedimentation or centrifugation.

2. The process as claimed in claim 1, in which the alkanol used is a primary alkanol.

3. The process as claimed in claim 2, in which the primary alkanol used is 1-decanol.

4. The process as claimed in claim 1, in which the aqueous base solution used in (D)(I) is sodium hydroxide solution, potassium hydroxide solution, a sodium or potassium hydrogencarbonate solution, a sodium or potassium carbonate solution or a mixture of two or more thereof and the aqueous acid solution used in (D)(II) is hydrochloric acid, sulfuric acid, phosphoric acid or a mixture of two or more thereof.

5. The process as claimed in claim 1, in which, in the re-extraction in (D)(I), a molar ratio of hydroxide ions to aminobenzoic acid is 1.0 to 5.0 and, in (D)(II), a molar ratio of hydronium ions to aminobenzoic acid is 1.0 to 5.0.

6. The process as claimed in claim 1, in which the pH is established in (B) and/or in (E.II)
(i) by adding an acid selected from hydrochloric acid, sulfuric acid or phosphoric acid or
(ii) by adding a base selected from aqueous ammonia, gaseous ammonia, sodium hydroxide solution, potassium hydroxide solution, a sodium or potassium hydrogencarbonate solution or a sodium or potassium carbonate solution, or by adding the fermentation process product containing aminobenzoate anions.

7. The process as claimed in claim 1, in which the extraction in (C) is performed at a temperature of 20° C. to 90° C.

8. The process as claimed in claim 1, in which the re-extraction in (D) is performed at a temperature of 20° C. to 90° C.

9. The process as claimed in claim 1, in which
the fermentable carbon-containing compound is selected from starch hydrolyzate, sugarcane juice, sugarbeet juice, hydrolyzates of lignocellulosic raw materials or a mixture of two or more thereof, and
the nitrogen-containing compound is selected from ammonia gas, aqueous ammonia, (at least) one ammonium salt, soya protein, urea or a mixture of two or more thereof.

10. The process as claimed in claim 1, in which the microorganisms are selected from Escherichia coli, Pseudomonas putida, Corynebacterium glutamicum, Ashbya gossypii, Pichia pastoris, Hansenula polymorpha, Kluyveromyces marxianus, Yarrowia lipolytica, Zygosaccharomyces bailii or Saccharomyces cerevisiae.

11. The process as claimed in claim 1, in which the second alcoholic phase, optionally after a purification, is recycled into the extraction of (C).

12. The process as claimed in claim 1, in which
(F) the first aqueous phase is separated by distillation into a third aqueous phase and a third alcoholic phase, where the third aqueous phase is disposed of as wastewater and the third alcoholic phase is recycled into the extraction of (C).

13. The process as claimed in claim 1, comprising (E.II).

14. The process as claimed in claim 1, in which ortho-aminobenzoic acid or para-aminobenzoic acid is produced.

15. The process as claimed in claim 1, in which the process product of the fermentation is freed of microorganisms before the crystallization in (B)(III).
